(19) United States
(12) Patent Application Publication
(10) Pub. No.: US 2025/0073197 A1
(43) Pub. Date: Mar. 6, 2025

(54) THE ANTI-INFLAMMATORY ROLE OF CANNABIDIOL, RETINYL PALMITATE, BETA-CARYOPHYLLENE AND TRIBUTYRIN WHEN ADMINISTERED IN CONJUNCTION

(71) Applicant: FLG Sciences, LLC, Miramar, FL (US)

(72) Inventors: Marcello Ferro, Miramar, FL (US); Adolfo Graubard, Weston, FL (US); Hector L. Lopez, Cream Ridge, NJ (US)

(21) Appl. No.: 18/821,273

(22) Filed: Aug. 30, 2024

Related U.S. Application Data
(60) Provisional application No. 63/579,567, filed on Aug. 30, 2023.

(51) Int. Cl. A61K 31/225; A61K 31/00; A61K 31/015; A61K 31/07; A61P 29/00

(52) U.S. Cl. CPC A61K 31/225; A61K 31/015; A61K 31/07; A61K 31/658; A61P 29/00

(57) ABSTRACT

Provided herein are methods and compositions for a synergistic treatment to improve pathological changes in diseases associated with metabolic syndrome, such as type 2 diabetes, fatty liver, non-alcoholic liver cirrhosis, fibromyalgia Alzheimer's disease and neurodegeneration. The synergistic combination of at least three of the four ingredients including CBD, BC and TB, as well as RP and BC.

### CROSS-REFERENCE TO RELATED APPLICATIONS

The present application claims priority U.S. provisional application Ser. No. 63/579,567, filed Aug. 30, 2023, herein incorporated by reference in its entirety.

### BACKGROUND

The invention generally relates to therapeutics.

Liver cirrhosis is the final common pathologic pathway of liver damage from a wide variety of chronic liver diseases. According to scientific publications, non-alcoholic fatty liver disease (NAFLD) is considered the hepatic phenotype of MetS, and its prevalence has been growing in parallel with the global epidemics of obesity and type 2 diabetes. The clinical spectrum of NAFLD ranges from simple steatosis to steatosis. -non-alcoholic hepatitis (NASH), which can progress to cirrhosis and hepatocellular carcinoma (HCC), report Asakawa et al. (2019). According to these authors, in addition to chronic hepatitis virus infection and alcohol consumption, much attention has been paid to NASH as the main cause of HCC in the next decade. Although several studies indicate the importance of fibrosis as the strongest determinant of HCC development, it is still unclear how simple steatosis progresses to NASH and what provides the carcinogenic tissue microenvironment. The prevalence of NAFLD in the US population is estimated at ˜24% (or ˜65 million) and up to a third of these have NASH. The prevalence of NAFLD is constantly increasing in parallel with the increase in the prevalence of obesity. NAFLD/NASH is already the third leading liver transplant indication and the second leading cause of HCC leading to liver transplantation in the US, and is likely to be the leading transplant indication by 2020 report Tsuchida et al. (2018). From a molecular point of view, MS-bound NAFLD/NASH occurs through the activation of a specific transcription factor, NF-κβ. This nuclear factor is a key transcriptional regulator of the inflammatory response and plays an essential role in the regulation of inflammatory signaling pathways in the liver. First, NF-κβ is activated in virtually all chronic liver diseases, including alcoholic liver disease, non-alcoholic fatty liver disease (NAFLD), viral hepatitis, and biliary liver disease. Second, NF-κβ regulates multiple essential functions in hepatocytes, Kupffer cells and hepatic stellate cells (HSCs). Third, genetic inactivation of different signaling components of NF-κβ results in liver phenotypes that include spontaneous injury, fibrosis and carcinogenesis, suggesting that NF-κβ makes an essential contribution to liver homeostasis and wound healing processes. NF-κβ has attracted wide attention among researchers from different fields. The central role in immunological processes and its apparent involvement in various diseases has been reported in several scientific studies to date. As NF-kB can be controlled by a family of peroxisome nuclear receptors (PPARs), activating these receptors with specific agonists seems to be a promising path in relation to the treatment of liver-associated pathologies. The problem is that synthetic exogenous PPARγ agonists trigger many side effects, as in the case of thiazolidinediones, including rosiglitazone or pioglitazone. Diarrhea, nausea, vomiting, stomach, sore throat, muscle pain, headache, loss of appetite, back pain, bloating, and weakness have been reported with the use of this drug (Table 1).

TABLE 1

| Drug | Action | Disease | Side effect | Authors |
|---|---|---|---|---|
| Rosiglitazone | PPARγ agonist | Diabetes | Edema | Day et al (2007) |
| Pioglitazone | PPARγ agonist | Diabetes | Edema | Smith (2001) |
| Balaglitazone | PPARγ agonist | Diabetes | Edema | Agrawal et al (2012) |
| Fenofibrates | PPARα agonist | Dyslipidemia | Pancreatitis | Farnier (2008) |
| Bezafibrate | PPARα agonist | Dyslipidemia | Hepatotoxicity | Monk & Todd (2008) |

The present invention attempts to solve these problems, as well as others.

### SUMMARY OF THE INVENTION

Provided herein are methods and compositions for a synergistic treatment to improve pathological changes in diseases associated with metabolic syndrome, such as type 2 diabetes, fatty liver, non-alcoholic liver cirrhosis, fibromyalgia Alzheimer's disease and neurodegeneration. The synergistic combination of at least three of the four ingredients including CBD, BC and TB, as well as RP and BC.

The methods and compositions are set forth in part in the description which follows, and in part will be obvious from the description, or can be learned by practice of the methods, apparatuses, and systems. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a graph showing the Cellular AOx Capacity in AML-12 liver cells of Vit A-beta-caryophyllene βC)-tributyrin (TriB), Vit A-βC-CBD, βC-CBD-TriB, and Vit A-βC, where all treatments were compared back to the CTL cells using dependent sample t-tests; no comparison between 3-hr versus 12-hr treatments were made; * indicates greater than control cells; and # indicates less than control cells.

FIG. 2A is a graph showing the Inflammatory signaling (pro-caspase 1) in AML-12 liver cells tested with Vit A-βC-TriB, Vit A-βC-CBD, βC-CBD-TriB, and Vit A-βC; FIG. 2B is a graph showing the Inflammatory signaling (pro-caspase 1) in C2CL12 muscle cells tested with Vit A-βC-TriB, Vit A-βC-CBD, βC-CBD-TriB, and Vit A-βC; notably, all treatments were compared back to the CTL cells using dependent samples t-tests; no comparison between 3-hr versus 12-hr treatments were made; #, indicates less than control cells.

FIG. 3 is a graph showing SOD2 protein levels in the C2CD12 muscle cells tested with Vit A-βC-TriB, Vit A-βC-CBD, βC-CBD-TriB, and Vit A-βC; all treatments were compared back to the CTL cells using dependent samples t-tests; no comparison between 3-hr versus 12-hr treatments were made; and *, indicates greater than control cells; #, indicates less than control cells.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present composition or formula comprising the synergistic combination of at least three of the four ingredients including CBD, BC and TB, as well as RP and BC. These combinations showed significant results in liver cells and muscle cells both in 3 h treatments and in 12 h treatments with TNF-alpha.

### Definitions

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. Pharmaceutical composition includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes). Exemplary prodrugs release the corresponding free acid upon cleavage, and the hydrolyzable ester-forming residues of the compounds of the present invention.

The term “pharmaceutically acceptable” refers to derivatives, analogues and salts which are physiologically acceptable for use in mammals, and which are not unduly toxic or otherwise unacceptable for such use. The term “mammals” includes human and non-human mammals, including domestic animals, e.g. cats, dogs, rodents, cattle, horses and the like, as well as non-domesticated animals.

The term “excipient” as used herein has its conventional meaning and refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

### Butyric Acid/Butyrate

Butyric acid or Butyrate is a short-chain fatty acid that acts as an energy source for intestinal epithelial cells and as a key mediator of various immune processes, modulating gene expression primarily through the inhibition of histone deacetylation. Through these effects, butyrate has been proposed for the treatment of many intestinal diseases as well as inflammation of the liver. Butyrate's functions range from being an energy source for colonocytes to being a key mediator of anti-inflammatory and antitumorogenic effects. Researchers such as Wang et al. (2012) and Guilloteau et al. (2010) had already shown that a reduced number of butyrate-producing bacteria were found in the colon of patients with ulcerative colitis (UC) and colon cancer. As the intestinal epithelium needs to maintain a low degree of inflammation so that it can always be prepared for constant immunological challenges on the mucosal surface, the immune control of these cells cannot suffer inflammatory and oxidative damage, as this can even develop cancer. Many studies have shown that butyrate can act as an anti-inflammatory agent. Several human and animal studies have reported that the pro-inflammatory cytokines IFN-γ, TNF-α, IL-1β, IL-6, and IL-8 are inhibited, while IL-10 and TGF-β are up-regulated in response to butyrate. The mechanism to which the anti-inflammatory effect of butyrate is attributed, at least in part, is due to the inhibition of NF-κβ activation. Several studies have suggested that butyrate suppresses NF-κβ signaling pathways, rescuing the redox machinery and controlling ROS, which mediate NF-κβ activation. Li et al (2012) reported that butyrate also demonstrated a mechanism of action related to promoting energy expenditure and inducing mitochondrial function through stimulation of the peroxisome proliferator-activated γ receptor (PGC)-1α coactivator. More recently, Rose et al. (2014) demonstrated that fibroblast growth factor (FGF) 21, which plays an important role in lipid metabolism, is induced by butyrate and involved in the stimulation of fatty acid β-oxidation in the liver, revealing the important role of this fatty acid in related to metabolic syndrome.

Tributyrin is a triglyceride and a butyrate ester that may be obtained by formal acylation of the three hydroxy groups of glycerol by butyric acid. As used herein, a derivative of tributyrin includes beta hydroxybutyrate, monobutyrin, dibutyrin, triacetin, tripropionate, glyceryl monoacetate, glyceryl diacetate, acetoacetate, a butyrate mono-ester, prodrug of butyric acid, a butyrate di-ester, and any derivatives thereof.

A pharmaceutically effective amount of butyrate may be administered via triglyceride tributyrin (e.g., glyceryl tributyrate or tributyrin). The butyrate via triglyceride tributyrin may be administered separately and/or in conjunction with one or more of the other described compounds (e.g., beta-hydroxybutyrate, fatty acids and/or esters, etc.). For example, up to approximately 200 mg/kg of the individual may be administered (e.g., up to 3 times daily). Administration of the tributyrin may allow a delayed release of butyrate to the body as the tributyrin is processed by the body of the individual. The tributyrin may be unencapsulated and/or encapsulated (e.g., microencapsulated). Tributyrin may be organic and non-organic salt cations.

Tributyrin (butyric acid prodrug)/butyric acid equivalents include Mineral salts selected from the group consisting of: butyrates, Sodium Butyrate, Magnesium butyrate, Potassium butyrate, Calcium butyrate, and organic and non-organic salt cations. Tributyrin (butyric acid prodrug)/butyric acid equivalents include Esters—butanoates selected from group consisting of: Butyl butyrate, Butyryl-CoA, Cellulose acetate butyrate, Estradiol benzoate butyrate, Ethyl butyrate, Methyl butyrate, and Pentyl butyrate. Tributyrin (butyric acid prodrug)/butyric acid equivalents include Amino Butyrate esters, Tripropionin.

### CBD

Cannabidiol (CBD) is one of over 100 cannabinoids present in the Cannabis sativa plant. The mechanisms of action of cannabinoids are mediated by the cannabinoid receptor on the cell surface. Two types of these cannabinoid receptors have been identified so far and both are members of the G protein-coupled receptor superfamily, CB1 and CB2. However, studies have shown that CBD also has interactions at several other nuclear receptors. Rimmerman et al. (2011) demonstrated that analysis of the Illumina gene array (containing 24,000 gene probes) revealed that CBD affected the expression of many genes. Gene transcripts (1204) were significantly regulated by CBD (680 were up-regulated and 524 down-regulated). Of these genes, 123 transcripts were upregulated and 38 genes downregulated 2-fold or more. DAVID analysis of gene products that were significantly up-regulated by CBD showed genes related to amino acid metabolism and glutathione activity, tRNA aminoacylation, molecular and ionic transport, as well as oxidative stress response. Thus, CBD has been suggested in therapies linked to chronic inflammation and associated pathologies, modulating its respective receptors. Several studies have shown that one of the main causes of neurological diseases as well as the metabolic syndrome begins with a chronic inflammatory process triggered by oxidative stress, say Uttara et al. (2011). As inflammation occurs from a molecular point of view through the activation of NF-κβ, this nuclear factor may have its metabolic activity reduced through PPARs. PPARs are ligand-activated receptors with distinct physiological functions in the regulation of lipid and glucose metabolism, as well as the inflammatory response]. Activation of PPARa and γ enables coordinated upregulation of numerous fatty acid oxidation enzymes (FAO), resulting in significant PPAR-driven increases in FAO mitochondrial flux. In a study with CBD, Ament et al. (2012) demonstrated that PPAR-γ can be activated by a specific agonist, CBD itself. CBD works as a PPAR-γ agonist, modulating inflammatory processes in cells through the activation of the peroxisome proliferator response element (PPRE) without the side effects present in similarly acting drugs.

It should be appreciated that in the context of the present invention the terms “cannabidiol compound”, “cannabidiol” or “CBD” (which may be used interchangeably unless the context clearly dictates otherwise) refer to any natural, semi-synthetic or synthetic cannabinoid compound.

CBD, unless a particular other stereoisomer or stereoisomers are specified, includes the compound “Δ²-cannabidiol.” These compounds are: (1) Δ⁵-cannabidiol (2-(6-isopropenyl-3-methyl-5-cyclohexen-1-yl)-5-pentyl-1,3-benzenediol); (2) Δ⁴-cannabidiol (2-(6-isopropenyl-3-methyl-4-cyclohexen-1-yl)-5-pentyl-1,3-benzenediol); (3) Δ³-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol), (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol).

CBD may include cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannahivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

According to one embodiment, the CBD compound comprises the following general Formula (I):

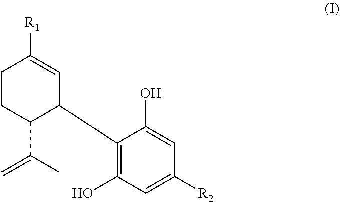

Wherein R1 is an alkyl; and

R2 is selected from a straight or branched alkyl having 5 to 12 carbon atoms; an —OR3 group, wherein R3 is a straight or branched alkyl having 5 to 9 carbon atoms or a straight or branched alkyl substituted at the terminal carbon atom by a phenyl group; or a —(CH₂)n-O-alkyl group, wherein n is an integer from 1 to 7 and the alkyl group has 1 to 5 carbons.

In one embodiment, R1 is CH₃ and R₂ is a straight alkyl having 5 carbon atoms (i.e. C₅H₁₁).

In another embodiment, the CBD compound is cannabidiol. CBD, the molecular formula of C₂₁H₃₀O₂, as depicted in the following formula (II):

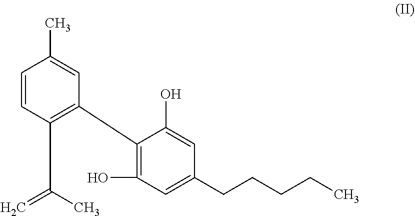 [SMILES: C=C(C)c1ccc(C)cc1-c1c(O)cc(CCCCC)cc1O]

The cannabidiol of formula (II) may be a natural cannabidiol obtainable by extraction from a plant member of the genus Cannabis or any preparations of Cannabis (e.g., processed plant material). According to one embodiment, the natural cannabidiol may be extracted from Cannabis sativa or one of its preparations, e.g., marijuana, hashish, etc. According to one embodiment, the natural cannabidiol can be extracted from Cannabis using methods such as described, for example, in U.S. Pat. No. 6,403,123, and in Gaoni and Mechoulam [J Chem Soc 93:217-224 (1971)], both incorporated herein by reference.

The cannabidiol may also be a synthetic cannabidiol or a derivative thereof which can be generated using methods such as those described, for example and without being limited thereto, in US 2003/166727, and incorporated herein by reference.

In some embodiments, the composition comprises a CBD derivative. The term “CBD derivative” as used herein means a CBD derivative having an anti-inflammatory effect, or an analgesic effect, or having an ameliorating effect on inflammatory disease, disorder or conditions; or alternatively, a CBD derivative that binds to CB(1) and/or CB(2) cannabinoid receptors.

In some embodiments, a CBD derivative is selected from (−)-7-hydroxy-CBD, which is known from WO 2015/198077 to reduce triglyceride levels and treat fatty liver; (−)-CBD-7-oic acid, which is known from Haj 2015 to have an anti-inflammatory effect; and the dimethylheptvl (DMH) homolog of CBD, which is known to have an anti-inflammatory effect (Ben-Shabat 2006; Juknat 2016), and the corresponding compounds in the enantiomeric (+)-CBD series.

In some embodiments, a CBD derivative is characterized by a structure, wherein at least one of the hydroxyl substituent groups is converted to a stable form thereof. In some embodiments, a CBD derivative is cannabinol comprising a quinone ring. In one embodiment, a CBD derivative is an endocannabinoid derivative. In another embodiment, the pentyl group on the phenyl ring of the CBD is replaced with any straight-chain or branched alkyl group selected from (C1-C18)alkyl, optionally substituted.

In some embodiments the CBD is prepared from a cannabis extract. In some embodiments the term “CBD or a derivative thereof” refers to between 80% and 99% pure CBD. In some embodiments the term “CBD or a derivative thereof refers to between 90% and 99% pure CBD. In some embodiments the term “CBD or a derivative thereof” refers to between 93% and 99% pure CBD. In some embodiments the term “CBD or a derivative thereof” refers to between 95% and 99% pure CBD. In some embodiments the term “CBD or a derivative thereof refers to between 95% and 97% pure CBD. In some embodiments the term “CBD or a derivative thereof” refers to about 97% pure CBD. All % hereinabove are weight %.

In some embodiments, the CBD or a derivative thereof is substantially and/or essentially devoid of tetrahydrocannabinol (THC). In some embodiments, a composition of the invention, as described herein, is substantially and/or essentially devoid of THC. In one embodiment, substantially and/or essentially devoid of THC means less than 10% by weight THC. In one embodiment, substantially and/or essentially devoid of THC is less than 7% by weight THC. In one embodiment, substantially and/or essentially devoid of THC is less than 5% by weight THC. In one embodiment, substantially and/or essentially devoid of THC is less than 3% by weight THC. In one embodiment, substantially and/or essentially devoid of THC is less than 1% by weight THC. In one embodiment, substantially and/or essentially devoid of THC is less than 0.5% by weight THC. In one embodiment, substantially and/or essentially devoid of THC is less than 0.3% by weight THC. In one embodiment, substantially and/or essentially devoid of THC is less than 0.1% by weight THC.

In some embodiments, the CBD is synthetically prepared.

### Beta-Caryophyllene

The term “beta-caryophyllene” is used herein to encompass the secondary metabolite, bicyclic sesquiterpene,-4, 11,11-trimethyl-8-methylene-bicyclo[7.2.0]undec-4-ene, which is present in, for example, plant-derived oleoresins, essential oils, solutes, distillates, extracts, fermentations, infusions and leaching, from plants including, but not limited to, Cannabis spp. including Cannabis sativa, Cannabis indica and Cannabis ruderalis, Humulus lupulus, Carum nigrum, Eugenia caryophyllata, Ocimum micranthum, Origanum vulgare, Piper guineense, Cinnamomum zeylanicum, Carthamus tinctorius, Helichrysum italicum, Copaifera spp. including Copaiba officinalis, Copaibaguianensis, Copaiba martii hayne, Copaiba Duckei, Copaiba reticulata, Copaiba multijuga, Copaiba confertiflora, Copaiba langsdorffii, Copaiba coriacea, Copaiba trapezifolia, Copaiba lucens, Copaiba paupera and Copaiba cearensis, Syzygium aromaticum (clove), piper nigrum (black pepper), Zingiber nimmoni, Zingiber officinale, Ocimum canum, Ocimum selloi, Piper cubeba, Aframomum melegueta, Panax ginseng, Zanthoxylum piperitum, Zanthoxylum simulans, Zanthoxylum bungeanum, Zanthoxylum rhesta, Zanthoxylum acanthopodium, Zanthoxylum piperitum, Syzgium aromaticum, Mentha longifolia, Ocimum tenuiflorum, Micromeria fruticosa, Salvia triloba, Salvia canariensis, Rosmarinus officinalis, Satureja thymbra, Satureja montana, Micromeria fruticosa subsp. Barbata, Piper longum, Piper retrofractum, Satureja obovata, Schinus terebinthifolius, Spilanthes acmella, Spilanthes oleracea, Persicaria hydropiper, Artemisia abrotanum, Persicaria odorata, Rhus coriaria, Xylopia aethiopica, Cymbopogon citratis, Pandanus amaryllifolius, Myrica gale, Myrica cerifera, Myrica pensylvanica, Origanum heracleoticum, Ocimum kilimandscharicum, Melissa officinalis, Mentha acquatica, Salvia officinalis, Salvia gillesii, Hyssopus officinalis, Thymus vulgaris, Teucrium cyprium, Teucrium divaricatum var. canescens, Artemisia salsoloides, Thymus zygis subsp. Sylvestris, Teucrium chamaedrys, Origanum minutiflorum, Ocimum basilicum, Thymus x citriodorus, Micromeria Juliana, Origanum onites, Origanum vulgare subsp. hirtum, Rosmarinus tomentosus, Lippia alba, Thymus riatarum, Rosemarinus eriocalyx, Ageratum conyzoides, Teucrium arduini, Teucrium kotschyanum, Nepeta racemosa, Rosmariunus x lavandulaceus, Thymus funkii, Coridothymus capitatus, Origanum syriacum, Thymus cilicicus, Eucalyptus porosa, Laurus nobilis, Daucas carota, Eucalyptus leucoxylon, Teucrium micropodioides, Leonotis leonurus, Micromeria varia subsp. thymoides, Hyptis suaveolens, Plectranthus coleoides, Vitex agnus-castus, Calamintha nepeta, Micromeria myrtifolia, Mentha aquatic, Salvia dorisiana, Ocimum suave, Sideritis scardica, Plectranthus incanus, Mentha x piperita, Hyssopus officinalis subsp. aristatus, Rosmarinus x mendizabalii, Satureja subspicata subsp. librnica, Sideritis mugronesis, Eucalyoptus fasiculosa, Teucrium gnaphalodes, Dictamnus albus, Satureja cilicica, Monardia citriodora, Sideritis germanicolpitana, Zingiber officinale, Eucalyptus sparsa, Thymus longicaulis, Origanum vulgare var. gracile, Minthostachys mollis, Monardia didyma, Salvia sclarea, Eucalyptus melanophloia, Elsholtzia blanda, Eucalyptus desquamate, Teucrium pseudoscorodonia, Eucalyptus cuprea, Sideritis pauli, Eucalyptus lansdowneana, Teucrium salviastrum, Teucrium scorodonia, Elsholtzia eriostachya var. pusilla, Sideritis athoa, Aralia cordata, Eucalyptus intertexta, Teurcrium oxylepis subsp. oxylepis, Cleaonia lusitanica, Satureja cuneifolia, Eucalyptus largisparsa, Eucalyptus odorata, Teurcrium polium var. valentinum, Eucalyptus behriana, Eucalyptus populnea, Teurcrium oxylepis subsp. marianum, Origanum vulgare var. viride, Eucalyptus ochrophloia, Eucalyptus viridis, Teucrium asiaticum, Thymus zygis, Lonicera japonica, Achillea millefolium, Aesculus hippocastanum, Agastache rugosa, Alpinia galangal, Anethum graveolens, Angelica archangelica, Annona squamosal, Apium graveolens, Artemisia absinithium, Artemisia annua, Artemisia capillaris, Bidens pilosa, Boswellia sacra, Camellia sinensis, Carum carvi, Centella asiatica, Chamaemelum nobile, Chrysanthemum parthenium, Chrysanthemum x morifolium, Cinnamomum aromaticum, Cinnamomum camphora, Cinnamomum verum, Citrus limon, Citrus paradise, Citrus reticulate, Citrus sinensis, Coleus barbatus, Coriandrum sativum, Croton eluteria, Croton lechleri, Ellettaria cardamomum, Ephedra sinica, Eruca sativa, Eucalyptus albens, Eucalyptus angulosa, Eucalyptus astringens, Eucalyptus blakelyi, Eucalyptus bosistoana, Eucalyptus botryoides, Eucalyptus brassiana, Eucalyptus camaldulensis, Eucalyptus ceratocorys, Eucalyptus cladocalyx, Eucalyptus dealbata, Eucalyptus diversicolor, Eucalyptus dolichorhyncha, Eucalyptus erythrandra, Eucalyptus forrestiana, Eucalyptus globulus, Eucalyptus grandis, Eucalyptus incrassate, Eucalyptus maculata, Eucalyptus maiden, Eucalyptus melliodora, Eucalyptus moluccana, Eucalyptus occidentalis, Eucalyptus oviformis, Eucalyptus polyanthemos, Eucalyptus puncata, Eucalyptus siderophloia, Eucalyptus sideroxylon, Eucalyptus stoatei, Eucalyptus tereticornis, Eucalyptus tetraptera, Foeniculum vulgare, Hedychium flavum, Houttuynia cordata, Lantana camara, Leptospermum scoparium, Lindera benzoin, Magnolia denudate, Matricaria recutita, Malaleuca altemifolia, Melia azedarach, Mentha arvensis var. piperascens, Mentha pulegium, Mentha rotundifolia, Mentha spicata, Montanoa tomentosa, Murraya koenigii, Myrciaria dubia, Myristica fragrans, Myrrhis odorata, Nepeta cataria, Ocimum gratissimum, Panax ginseng, Pelargonium citrosum, Perilla frutescens, Petroselinum crispum, Pimenta dioica, Pimenta racemosa, Pimpinella anisum, Pinus strobus, Piper nigrum, Pistacia lentiscus, Populus tacamahacca, Psidium guajava, Ptychopetalum olacoides, Ravensara aromatic, Sambucus nigra, Vaccinium myrtillus, Sassafras albidum, Satureja hortensis, Stevia rebaudiana, Illicium verum, Gossypium sp., Tagetes filifolia, Tagetes lucida, Tagetes minuta, Tamarindus indica, Tanacetum parthenium, Teucrium polium, Trifolium pretense, Valeriana officinalis, Zea mays, Piper betel, Pycnanthemum tenuifolium, Thymus serpyllum, Pycnanthemum setosum, Pycnanthemum pycnanthemoides, Pycnanthemum virginianum, Thymus orospedanus, Pycnanthemum clinopodioides, Pycnanthemum loomisii, Pilocarpus microphyllus, Hedeoma hispida, Lavandula x intermedia, Cymbopogon nardus, Pycnanthemum pilosum, Cuminum cyminum, Pycnanthemum verticillatum, Thymus capitatus, Pycnanthemum muticum, Lepechinia calycina, Aloysia citrodora, Dracocephalum thymiflora, Leonurus cardiac, Lepechinia schiediana, Scutellaria galericulata, Hedeoma pulegioides, Micromeria croatica, Pycnanthemum californicum, Cunila origanoides, Pycnanthemum torreyi, Thymus mastichina, Lycopus europeus, Moldavica thymiflora, Juniperis communis, Satureja vulgaris, Elsholtzia polystachya, Lycopus virginicus, Scutellaria churchilliana, Pycnanthemum montanum, Agastache foeniculum, Agastache nepetoides, Carthamus tinctorius, Dracocephalum parviflora, Pycnanthemum beadle, Scutellaria parvula, Echinacea spp, Galeopsis tetrahit, Satureja douglasii, Balotta nigra, Ribes nigrum, Isanthus brachiatus, Moldavica parviflora, Elsholtzia cristata, Elsholtzia pilosa, Myrtus communis, Cordia verbenacea, Ferula galbaniflua, Commiphora gileadensis, Populus balsamifera, Citrus bergamia, Tanacetum annum, Abies balsamea, Ocimum basilicum ct linalool, Mentha citrate, Picea mariana, Malaleuca leucadendron var. cajuputi, Eriocephalus punctulatus, Cymbopogon winterianus, Pinus nigra laricio, Cupressus sempervirens, Psudotsuga menzies, Canarium luzonicum, Eucalyptus citriodora, Eucalypotus dives, Eucalyptus radiata, Agonis fragrans, Bowsellia carterii, Pelargonium roseum x asperum, Helichrysum bracteiferum, Helichrysum gymnocephalum, Helichrysum odoratissimum, Tsuga Canadensis, Malaleuca teretifolia, Citrus hystrix, Kunzea ambigua, Larix laricina, Lavendula angustifolia, Lavendula officinalis, Cymbopogon citradis, Cymbopogon citratus ct rhodinol, Citrus aurantifolia, Bursera delpechiana, Origanum marjorana, Litsea cubeba, Citrus aurantium var. amara, Malaleuca quinquenervia et 1,8 cineole, Pinus resinosa, Cymbopogon martini var. motia, Pogostemom cablin, Citrus aurantium var. bigardia, Pinus edulis, Pinus ponderosa, Cinnamomum camphor act 1,8 cineole, Rhododendron anthopogon, Rose damascena, Rosa damascena/Pelargonium Roseum x asperum, Rosmarinus officinalis ct camphor, Rosmarinus officinalis ct verbenone, Aniba rosaeodora, Cinnamosma fragrans, Pinus sylvestris, Abies sibirica, Abies alba, Lavandula latifolia, Hypericum perforatum, Cinnamomum glaucescens, Cinnamomum tamala, Thymus zygis, Thymus vulgaris ct linalool, Ocimum sanctum ct eugenol, Thymus vulgaris ct thymol, Curcuma longa, Picea glance, Zanthoxylum armatum, Cananga odorata, Ocimum mircanthum, Ocimum selloi, Citrus junos, and all Plantae taxa thereof, including, life, domain, kingdom, phylum, class, order, family, genus, species, super-species, sub-species, varieties, hybrids and chemotypes, phenotypes and genotypes, whether naturally occurring or genetically modified. Preferred plant sources of beta-caryophyllene include at least about 20% beta-caryophyllene, such as, at least about 25%, 30%, 35%, 40%, 45% or 50% beta-caryophyllene. Copaiba/Copaifera sp., cloves, and cinnamon, as well as herbs like oregano, basil, hops, C. sativa/indica and rosemary, are known to exhibit high concentrations of caryophyllene.

Functionally equivalent derivatives, analogues or salts of beta-caryophyllene, which are pharmaceutically acceptable, may replace beta-caryophyllene in the present composition. The term “functionally equivalent” as used with respect to derivatives, analogues and salts of beta-caryophyllene, refers to compounds which possess the activity or function of beta-caryophyllene, at least in part, to treat pain and/or inflammation.

Functionally equivalent derivatives or analogues, including structural and functional analogues, of beta-caryophyllene include compounds derived from beta-caryophyllene or a precursor thereof, including isomers thereof. Examples of functionally equivalent derivatives or analogues include, but are not limited to, alpha-humulene, 9-epi-(E)-Caryophyllene, [−]-Caryophyllene oxide or (−)-Epoxycaryophyllene, (1i?,4i?,6 ?,10S)-9-Methylene-4,12,12-trimethyl-5-oxatricyclo[8.2.0.0]Caryophyllene, 9-epi-Caryophyllene or (E)-Caryophyllene, epi-, cis-Caryophyllene, (+)(E)-Caryophyllene or 2-epi-(E)-O-Caryophyllene, and Isocaryophyllene (or (Z)-Caryophyllene or β-cis-Caryophyllene or (Z)-Caryophyllene or Bicyclo(7.2.0)undec-4-ene, 4,11,11-trimethyl-8-methylene-, (1R,4Z,9S)- or cis-Caryophyllene or γ-Caryophyllene or [1R-(1R*,4Z,9S*)]-4,11, 11-trimethyl-8-methylenebicyclo[7.2.0]undec-4-ene).

Functionally equivalent salts which are pharmaceutically acceptable salts of beta-caryophyllene are also encompassed herein for use to treat inflammation. The term “salts” refers to salts or esters of beta-caryophyllene that retain the desired biological activity of the parent compound to treat pain and/or inflammation, at least in part. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Although various natural sources of beta-caryophyllene exist which may be incorporated into the present composition, it will be appreciated that beta-caryophyllene for use in the present composition may also be synthetically derived.

Beta-Caryophyllene (BC) is the main constituent of the essential oil of Cannabis Sativa L and many other plants such as cinnamon (Cinnamomum spp.), oregano (Origanum vulgare L.) and black pepper (Piper nigrum L.), and mainly in copaiba. It has a wide range of pharmacological actions, including analgesic, antimicrobial, antifungal, antioxidant and anti-inflammatory activities. BC functions as a PPAR-α agonist. Interestingly, PPAR-α is predominantly present in many cells, including the liver. Chen et al. (2015) demonstrated that PPAR-α plays a critical role in modulating energy balance and regulating hepatic lipid metabolism. A synthetic PPAR-α ligand, fenofibrate, works to reduce serum triglyceride levels and is used clinically to improve plasma lipid disorders at risk for cardiovascular disease. Some clinical studies have shown that fenofibrate also plays a key role in maintaining normal liver function and improving insulin resistance in patients with nonalcoholic fatty liver disease, but with unpleasant side effects. Furthermore, several observations have indicated that PPAR-α may also play a key role in the molecular control of fibrogenesis, states Varga (2018). PPAR-α has also been shown to be significantly involved with inflammation, as PPAR-α activation protects against hepatic ischemic reperfusion injury in mice. More recent studies add to a growing body of evidence that PPAR-α may be a promising therapeutic target in physiological and pathological processes involved in liver disease (Oncotarget. 2015 Dec. 15; 6(40): 42530-42540. Chen L.). Varga (2018) also showed that, given the safety of BC in humans, this food additive has a high translational potential in the treatment or prevention of liver damage associated with oxidative stress, inflammation and steatosis. Another inflammatory protection mechanism exerted by BC is the attenuation of pro-inflammatory cytokines and inflammatory mediators such as cyclooxygenase-2 (COX-2) and nitric oxide synthase (iNOS), also restoring antioxidant enzymes, especially superoxide dismutase (SOD), inhibiting lipid peroxidation as well as glutathione (GSH) depletion. Ojha et al. (2016) demonstrated that BC from caopaiba provided neuroprotection against rotenone-induced Parkinson's disease and the neuroprotective effects can be attributed to its potent antioxidant and anti-inflammatory activities. Due to its favorable taste and aroma and apparent lack of toxicity, BC is FDA approved as a flavoring food additive. BC, which is devoid of psychoactive effects, has been shown to activate CB2 cannabinoid receptors, which are primarily expressed on immune cells and are derived from the immune system. This makes BC a promising food-derived agent that can be therapeutically exploited to treat various inflammatory diseases, say Gertsch et al. (2008). BC has been reported to exert protective effects in experimental animal models of pain and inflammation (Gertsch et al., 2008), kidney injury (Horvath et al., 2012), ischemic stroke (Choi et al., 2013), disease (Ojha et al., 2016), toxic hepatitis (D-galactosamine and endotoxin-induced) (Cho et al., 2015), experimental liver fibrosis (Mahmoud et al., 2014) and colitis (Bento et al., 2015). 2011). BC has also recently been proposed as having anti-addictive potential (Al Mansourietal, 2014). BC has been reported to act on targets other than CB2 receptors, including sirtuin 1 (SIRT-1; Zheng et al., 2013), PPAR-α (Wuetal., 2014), fatty acid amide hydrolase (FAAH) or COX-2 (Chicca et al., 2014). Studies reported that BC treatment resulted in a decrease in the infarcted region, a decrease in the level of cardiac markers in the serum, an improvement in the ECG and a reversal of blood pressure, indicating the protective effects of this treatment and the significant improvement in the functional recovery of the myocardium.

### Vitamin A

Retinyl palmitate and beta-carotene or known as Vitamin A. Vitamin A deficiency has affected an expressive number of children, pregnant women and the low-income population. Approximately 125-130 million preschool-age children suffer from vitamin A deficiency. Approximately 7 million pregnant women in low-income countries also suffer from vitamin A deficiency, which is the leading cause of preventable pediatric blindness in the world in development. Deficiency of this vitamin also increases susceptibility to serious infections resulting in an increased risk of mortality in children and pregnant and lactating women. The most common cause of vitamin A deficiency is insufficient dietary intake, which is normally found in animal foods such as preformed vitamin A (or retinol) and in plant foods such as provitamin A. According to West et al. (2013), in developing countries, the widespread consumption of mostly plant-based diets exacerbated vitamin A deficiency due to the low bioavailability of provitamin A carotenoids [6,7]. While there has been considerable progress in controlling vitamin A deficiency worldwide [8], there is still a need for additional prevention efforts in the form of dietary diversification, fortification, and supplementation. Low levels of vitamin A (Retinyl palmitate) were observed in patients with stage III and IV head and neck cancer, as well as in those with secondary primary, suggesting a possible role of vitamin A deficiency as a cause of head and neck cancer. In animal studies Lotan (1997) found that natural and synthetic metabolites and analogues of vitamin A (retinoids) suppress head and neck and lung carcinogenesis. However, the precise mechanism of vitamin A and its derivatives such as carotenoids in preventing head and neck cancer is not fully understood. These agents are believed to restore the expression of genes that regulate cell growth and differentiation. Most of these effects are mediated by nuclear retinoic acid receptors (RARα, β and γ) and retinoid X receptors (RXRα, β and γ), which function as retinoid-activated transcription factors, states Youssef et al. 2004). In fact, retinoid receptors, especially the RXR, play a crucial role with PPAR receptors as heterodimers in the activation of PPRE in DNA. For this reason, recruiting ingredients that can interact as receptor agonists with synergistic actions is a way to go.

Retinyl palmitate, an ester form of retinoic acid (Vitamin A), and precursor of 9-cis retinyl palmitate, or derivatives of Vitamin A include, but not limited to, retinol palmitate, retinyl palmitate, retinol acetate, beta-carotene or combinations thereof. Alternatively, the retinoid may be all-trans-, 9-cis-, 11-cis retinoic acid. Retinyl palmitate may include trans-Retinyl palmitate, cis-retinyl palmitate, 2-retinyl palmitate, 3-retinyl palmitate, or combinations thereof. Vitamin A or retinyl palmitate may also include retinoid receptor agonist including AM-580, BMS641, BMS961, CD666, TTNPB, ATRA, Ro25-7386, methoprene acid or combinations thereof. Natural and non-natural derivative of retinoids or retinyl palmitate are also contemplated. The skilled artisan may determine what derivatives of retinoids or retinyl palmitate have similar activity to naturally occurring compounds. Other agonists that may activate the receptors RAR/RXRα, β or γ are also contemplated by the present invention. All retinoic acid/alpha/beta-carotene congeners & retinyl esters that are ligands & signal via RXR receptors are contemplated herein.

Vitamin A equivalents include Beta/alpha carotene, Beta-cryotoxanthin, Lutein, Zeaxanthin, Lycopene, Tazarotene (brand name: Tazorac), Adapalene, and Retinyl esters selected from the group consisting of: Palmitic acid, Oleic acid, Stearic acid, and Linoleic acid.

### Astaxanthin

Astaxanthin (ATX)—ATX, a red pigment that belongs to the xanthophyll subclass of carotenoids, has a strong antioxidant capacity and can scavenge singlet oxygen (O2-) as well as hydrogen peroxide (H₂O₂) and lipid peroxidation, Yuan et al. al. (2011). Due to its antioxidant capacity and cell signaling modulating properties, ATX has a variety of beneficial biological activities and effects, including protection against UV damage (Ito et al. 2018), anti-inflammatory and immunomodulatory activity (Park et al. 2010), alleviation of metabolic syndrome (MS) (Hussein et al. 2007), cardioprotective effects (Fasset & Coombes 2012), antidiabetic activity (Landon et al. 2020), prevention of neuronal damage (Galasso et al. 2018), anti-aging (Tominaga et al. 2017) and anticancer activity (Hormozi et al. 2019), as well as inhibition of cell membrane peroxidation (Kidd 2010). In general, ATX has an inhibitory effect on the development of diseases mainly associated with oxidative stress and mitochondrial dysfunction. Since mitochondria are a source of ROS, this can be a target in pathological conditions, states Kim (2019). ATX can reduce oxidative stress and maintain mitochondrial integrity. Sztrety et al (2019) proved that ATX supports mitochondrial function by protecting the redox balance in this organelle. ATX significantly reduces the physiologically occurring oxidative stress and keeps mitochondria in a reduced state, even after stimulation with H₂O₂. It also prevents loss of mitochondrial membrane potential in electron escape and increases mitochondrial oxygen consumption. ATX can also prevent mitochondrial dysfunction by permeating and co-localizing within mitochondria. In an in vivo study of geriatric dogs, Park et al. (2013) demonstrated that oxidative damage was mitigated and impaired mitochondrial function was restored in ATX treatment. In this study, the authors suggest that ATX prevents aging by increasing mitochondrial efficiency in ATP production and in the activity of the respiratory chain complex. ATX has also demonstrated its ability to inhibit the release of cytochrome c resulting from mitochondrial permeabilization and thus prevent mitochondria-mediated apoptotic cell death, state Baburina et al. (2019). In another study with myocardial cellulose, Fan et al. (2017) suggested that ATX inhibited cytochrome c release and myocardial cell apoptosis, decreasing ROS levels and the consequent formation of protein oxidation products and restoring mitochondrial membrane potential. Likewise, ATX elevated PGC-la levels in skeletal muscle, induced a decrease in plasma fatty acids during exercise, and prevented the reduction in intermuscular pH due to exercise. AXT has been shown to elevate the expression of PGC-la and its downstream proteins, which are involved in the activation of mitochondrial biogenesis, leading to acceleration of fat utilization during exercise through aerobic metabolism in mitochondria. Therefore, the elevation of PGC-la by ATX can induce the acceleration of lipid metabolism during exercise. Indeed, cytochrome c, a component of the mitochondrial electron transport chain and one of the main proteins inducible by PGC-la, was also upregulated by ATX.

Astaxanthin's formula is C₄₀H₅₂O₄ and is referred to as 3,3′-dihydroxy-4,4′-diketo-β,β′-carotene widely existed in nature, and is an important substance of carotenoid, has a higher physiological activity than other carotenoid. The molecular formula is as follows:

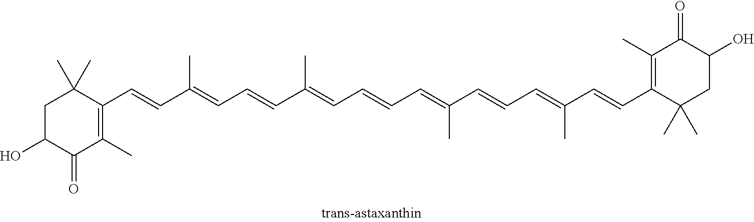 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

As for a kind of natural preparation, astaxanthin has broader uses and development prospects in various industries such as pharmaceuticals, aquaclutures, foods, chemistries.

Astaxanthin has multiple double bonds in its molecular structural formula, correspondingly has cis- and trans-isomers. Generally speaking, all trans-isomers astaxanthin has the highest activity relative to cis-isomers, astaxanthin has two isomers as 9-cis and 13-cis, because of influences of spatial structure.

In addition, due to four of chiral centers existed in its molecular structural formula, astaxanthin also has optical isomers. The main optical isomer includes three forms such as (3 R, 3 R)′—, (3 R, 3 S,′ meso)- and (3 S, 3′S)—, and the molecular structure of the three isomers are shown below. Different source's astaxanthin has different optical isomers. Different optical isomer has different physiological activities. Generally speaking, the bioavailability of (3 S, 3′S)-configuration in animal body is relatively high and is 15 wt % higher than the bioavailability of (3 R, 3 R)′-configuration, The molecular structural formula of astaxanthin of salmon is (3 S, 3 'S)-configuration,

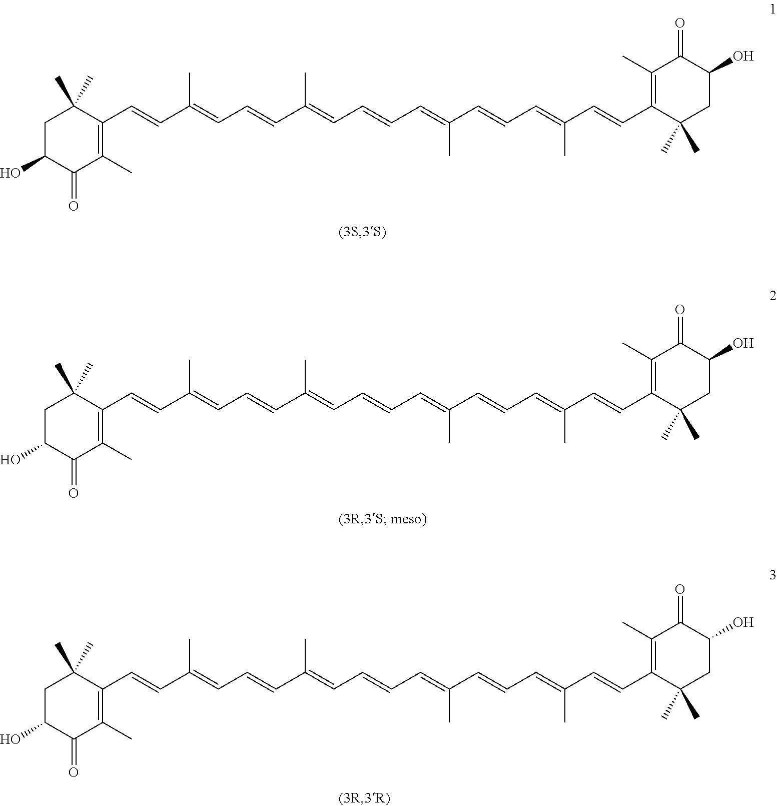

At present, astaxanthin is mainly produced in two ways such as chemical synthesis and natural extraction. Astaxanthin prepared by chemical synthesis is expensive and has significant differences in molecular structures, biological functions, application effects and biological safeties. Synthetic astaxanthin is a mixture of three different configurations wherein most of them is (3R and 3′S), Synthetic astaxanthin obviously has stabilities and antioxidant activities lower than that of natural astaxanthin, and the effects of biological absorption and pigmentation of synthetic astaxanthin are also worse than that of natural astaxanthin. And synthetic astaxanthin cannot be converted into natural astaxanthin in animal bodies. On the other hand, pollutions are inevitably produced in process of synthesis, and then leads to generation of some potential safety hazard and reduction of safety performance.

Embodiments may be further directed to pharmaceutical compositions comprising combinations of structural carotenoid analogs to said subjects. The composition of an injectable structural carotenoid analog of astaxanthin may be particularly useful in the therapeutic methods described herein. In yet a further embodiment, an injectable astaxanthin structural analog is administered with another astaxanthin structural analogs and/or other carotenoid structural analogs, or in formulation with other antioxidants and/or excipients that further the intended purpose. In some embodiments, one or more of the astaxanthin structural analogs are water soluble. The carotenoids derivative or analog of the invention have a structure chosen among

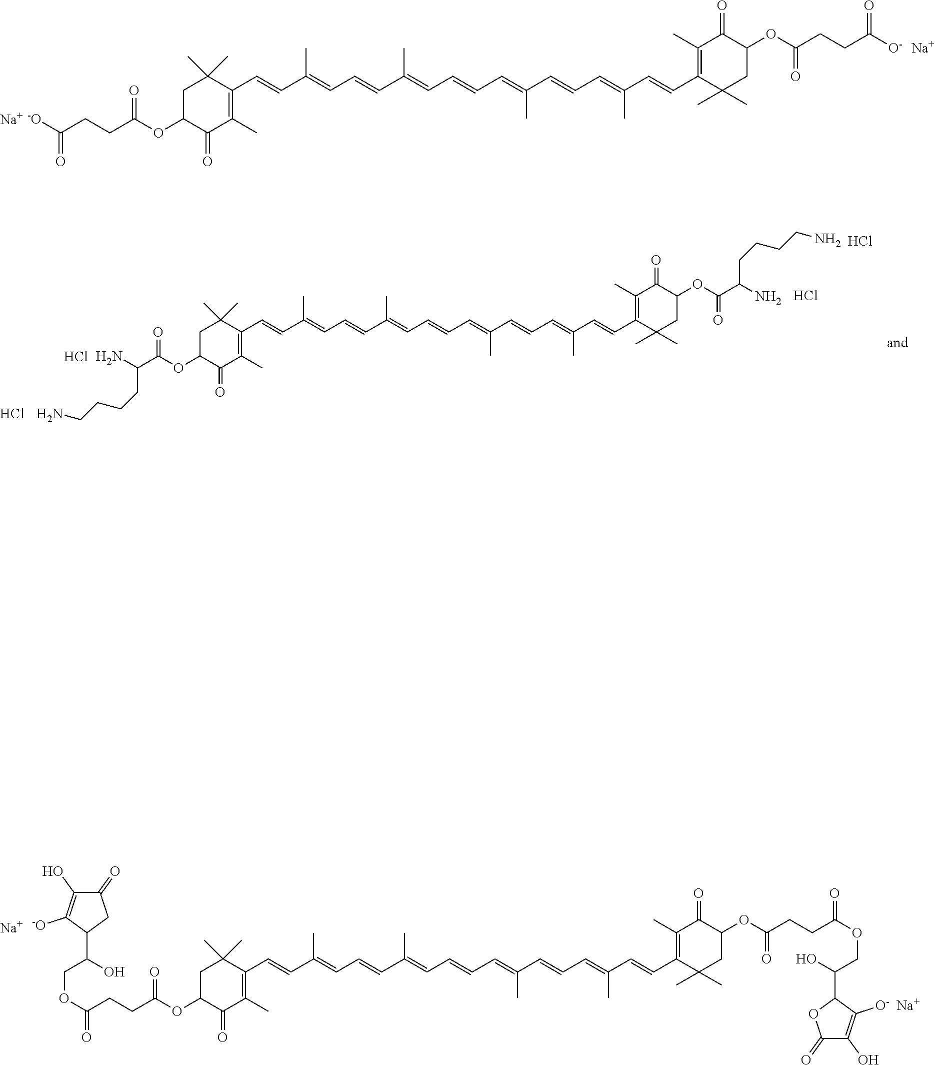

These carotenoid derivatives may be used in a pharmaceutical composition. In one embodiment, a pharmaceutical composition that includes these carotenoid structural analogs may be used for treating reperfusion injury.

As used herein, the terms “disodium salt disuccinate astaxanthin derivative”, “dAST”, “Cardax”, “Cardax™”, “rac”, and “astaxanthin disuccinate derivative (ADD)” represent varying nomenclature for the use of the disodium salt disuccinate astaxanthin derivative in various stereoisomer and aqueous formulations, and represent presently preferred but nonetheless illustrative embodiments for the intended use of this structural carotenoid analog. The diacid disuccinate astaxanthin derivative (astaCOOH) is the protonated form of the derivative utilized for flash photolysis studies for direct comparison with non-esterified, “racemic” (i.e., mixture of stereoisomers) astaxanthin. “Cardax-C” is the disodium salt disuccinate di-vitamin C derivative (derivative XXIII) utilized in superoxide anion scavenging experiments assayed by electron paramagnetic resonance (EPR) imaging.

### Lycopene

Lycopene, also known as psi-carotene, belongs to the carotenoid family. Carotenoid is a fat-soluble pigment synthesized by plants and microorganisms. The carotenoid comprises over 700 compounds and is responsible for the yellow, orange and red colors in many fruits and vegetables. Approximately 90% of carotenoids in the diet and in the human body are represented by 0-carotene, α-carotene, lycopene, lutein and cryptoxanthin. Epidemiological investigations indicate that lycopene has potential antioxidant properties, is able to scavenge ROS and alleviates oxidative stress in type 2 diabetic patients. The antioxidant properties of lycopene have been attributed to its highly conjugated double bonds and to a lesser influence of the presence of cyclic or acyclic end groups in the structure. Much scientific evidence has reported the link between lycopene and diabetes-induced oxidative stress, measuring various biomarkers and lipid peroxidation products, including endogenous enzymatic antioxidants such as glutathione peroxidase (GPx), superoxide dismutase (SOD) and malondialdehyde (MDA) levels.) in plasma as well as in tissue samples, say Rani et al (2014). The antioxidant property of lycopene has been the main focus of research. According to Olempska-Beer (2006), among carotenoids, lycopene is reported as the most efficient singlet oxygen inhibitor. Cantrell et al. (2003) demonstrated that the extinction rate of lycopene was two times higher compared to β-carotene and 10 times higher compared to α-tocopherol. A comparison of the quenching ability relative to lycopene and other carotenoids is described as: lycopene>y-carotene>astaxanthin>canthaxanthin>α-carotene>β-carotene>bixin>zeaxanthin>lutein>cryptoxanthin>crocin>α-tocopherol>acid lipoic>glutathione.

Lycopene (ψ, ψ-carotene), the main carotenoid in tomatoes and tomato products, has excellent singlet oxygen quenching activity and free radical scavenger activity. Indicates. Lycopene also exhibits growth inhibitory effects on several types of human cancer cells and anti-carcinogenic effects on carcinogenesis in mice (Non-Patent Documents 5 to 8). Furthermore, epidemiological studies have shown that lycopene uptake is reduced in prostate cancer (Non-Patent Documents 9 to 12).

The lycopene that the present invention describes can comprise the isomers that one or more are different. For example, lycopene can comprise at least 10%, at least 20%, at least 30%, at least 40%, at least 50%, at least 60%, at least 70%, at least 80%, at least 90% or at least 95% (Z)-isomers, (all L)-isomers or cis-isomer, 5-cis- or 9-cis- or 13-cis-isomers for example, it has the bioavailability of improvement with respect to transisomer. Transisomer can be isomerizated into cis in vivo or in storage and processing procedure.

For example, the lycopene derivative with the biological property that is similar to lycopene can comprise carotenoid for example vitamin A acid, synthetic acyclic vitamin A acid; Or 1-HO-3′, 4′-two dehydrogenation lycopenes, 3,1′-(HO) 2-gamma carotene, 1,1′-(HO) 2-3,4,3′, 4′-four dehydrogenation lycopenes and 1,1′-(HO) 2-3, the two dehydrogenation lycopenes of 4-.

The lycopene compound that the present invention describes can be natural, promptly derives from natural origin, for example, extracts from the plant of for example tomato or muskmelon. A series of extractions from plant, method concentrated and/or purification lycopene compound are known in the art. For example, can use the solvent extraction method that utilizes ethanol, DMSO, ethyl acetate, hexane, acetone, soybean or other plant oil or non-plant oil.

The lycopene compound that the present invention describes can synthesize, and promptly for example generates by chemical synthesis by artificial means. The method of method chemical synthesis lycopene and other carotenoid is known in the art. For example, can use to be used for the synthetic three step chemical synthetic methods of carotenoid, wherein generate the C of carrene (DCM) based on standard Wittig olefination reaction process ₁₅The organic solution of Jia Wan phosphonium sulfonates and the C of toluene ₁₀The organic solution of dialdehyde, and these two kinds of organic solutions are to combine with sodium methoxide solution gradually and experience condensation reaction to form the lycopene crude product. Then, use the described thick lycopene of routine techniques purifying, for example by adding glacial acetic acid and deionized water to described mixture, vigorous stirring makes water separate with organic facies, and extracts the organic facies of the lycopene crude product that contains in DCM and the water, Methyl alcohol is added in the organic facies, and DCM under reduced pressure removes through the way of distillation, Heat described crude product methyl alcohol lycopene solution then, and be cooled to slurry, filter and wash this slurries with methyl alcohol. Then, described lycopene crystal recrystallization and dry under hot nitrogen. Synthetic lycopene also can obtain from supplier (for example BASF Corp, NJ USA).

With respect to natural lycopene, synthetic lycopene can comprise cis-isomer that ratio increases. For example, synthetic lycopene can reach 25% 5-cis, 1% 9-cis, 1% 13-cis and other cis-isomers of 3%, and by the lycopene that tomato generates may be 5-cis, the 0-1% of 3-5% 9-cis, 1% 13-cis and <other cis-isomers of 1%. Because with respect to trans lycopene, the bioavailability of the lycopene of cis has increased, therefore in some embodiments, preferred synthetic lycopene.

Above-mentioned lycopene derivative can generate by the chemical synthesis that is similar to above-mentioned synthetic method, or generates from the natural lycopene that plant material extracts by chemical modification.

In preferred embodiment, described non-hydrophilic antioxidant lycopene is prepared with lactalbumin. Lycopene formulations with lactalbumin (being called ‘lactolycopene’) is known (for example to see J. Nutr.132:404-408 such as Richelle in this area, 2002 and PCT/EP01/06145) and commercially available (INNEOV, L′ Or é al (UK) Ltd, London), Lactolycopene can obtain by the method that the described method of these documents or the present invention describe.

Recently, some of the oxidative metabolites of lycopene with unique five-membered ring end groups, namely 2,6-cyclolycopene-1,5-epoxide (1,5-epoxy-1,2,5,6-tetrahydro)-2,6-cyclo-ψ, ψ-carotene-1′, 5′-diol), 2,6-cyclolycopene-1,5-diol (1,2,5,6-tetrahydro-2,6-cyclo)-Ψ, ψ-carotene-1′, 5′-diol) and 1,16-didehydro-2,6-cyclolycopen-5-ol (1,16-didehydro-1,2,5,6-tetrahydro)-2,6-cyclo-ψ, ψ-carotene-1′, 5′-diol) from human serum, milk, tomato and tomato products (Non-patent Documents 13 to 18).

However, the anticancer activity of the conventionally known lycopene is by no means high. Three new oxidative metabolites derived from lycopene: (erythro)-lycopene-5,6-diol, (threo)-lycopene-5,6-diol, and 1,16-dehydro-2,6-cyclo Lycopen-5-ol B and four new oxidative metabolites derived from γ-carotene, namely 2′, 6′-cyclo-γ-carotene-1′, 5′-diol A, 2′, 6′-Cyclo-γ-carotene-1′, 5′-diol B, (erythro)-γ-carotene-5,6-diol, and (threo)-γ-carotene-5,6-diol are gack (Momodica cochinchinensis)), And the structure of these new oxidative metabolites was determined based on spectroscopic data, revealing their outstanding anticancer activity.

The terms such as “carotenoid analog” and “carotenoid derivative,” as used herein, generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically below.

“Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

### Lutein

Lutein & Zeaxanthin—Zeaxanthin and lutein are two carotenoids that assume a different position in biological membranes than β-carotene. Aeaxanthin and lutein have polar and nonpolar regions that line up with the respective polar and nonpolar regions of the phospholipid bilayers, whereas β-carotene is fully nonpolar and dissolves in the membrane core. In both plants and humans, both zeaxanthin and lutein have multiple functions, including photoprotection against damage from intense light, detoxification of oxidants (reactive oxygen species, ROS, and other radicals), and maintenance of structural and functional integrity of biological membranes. Despite similar qualitative roles, effects differ in degree between zeaxanthin and lutein. Zeaxanthin is the most effective antioxidant, presumably due to its longer system of conjugated double bonds and has a more pronounced impact on membrane integrity. Humans and other animals cannot synthesize carotenoids, and must obtain them from the diet. In humans, the best-studied role of carotenoids is in vision. Much of the research on zeaxanthin and lutein in humans so far has focused on their role in photoprotection against damage, especially age-related blindness. Both age-related macular degeneration and Alzheimer's disease are pro-inflammatory diseases that involve immune system dysfunction and uncontrolled inflammation. Demmig-Adams et al. (2020) suggested that the perception that a state of chronic low-grade inflammation plays a key role in a number of related chronic diseases such as cardiovascular disease, diabetes, certain cancers, autoimmune diseases, and disorders such as anxiety, depression, bipolar disorder, schizophrenia, post-traumatic stress disorder have been very well documented in the last decade. Surprisingly, memory, attention, learning, and overall cognitive performance are also impaired by systemic inflammation even in adults who are considered healthy and not diagnosed with a disease or disorder, say Ashraf-ganjouei et al. (2020). It can be hypothesized that carotenoids with antioxidant and anti-inflammatory functions may improve some or all of these conditions. There is a substantial body of evidence for correlations between higher levels of carotenoids and a lower risk of various pro-inflammatory diseases in several clinical studies. While more research is needed to assess the question of causality between xanthophylls and a decrease in systemic inflammation, evidence of a causal relationship is beginning to emerge from manipulative studies in animal and human models. For example, Zhou et al. (2017) reported that long-term zeaxanthin supplementation reduced levels of pro-inflammatory hormones and decreased diabetic symptoms, anxiety, and depression in diabetic rats. Stringham et al. (2019) extended these findings to humans and, in particular, healthy young subjects who were supplemented with zeaxanthin, meso-zeaxanthin, and lutein for six months. The results of this study included significantly lower levels of pro-inflammatory hormones and improved cognitive performance on a variety of complex tasks, including processing speed, as well as various aspects of memory and attention, the authors state. Similar improvements in cognitive function in healthy young adults as a result of zeaxanthin and lutein supplementation were reported in the clinical studies by Renzi-Hammond et al. (2017).

### Description of Embodiments

Diseases are always associated with two factors, oxidative stress and inflammation. The onset of pathologies is always a result of these two factors. The conjugated ingredients used in the present formula are focused on inhibiting both oxidative stress and stopping chronic inflammatory processes. When reactive oxygen species (ROS) reach levels over which cells no longer have control, apoptosis mechanisms end up coming into action leading to tissue death and/or disease. This is very common in nonalcoholic liver cirrhosis. Therefore, one embodiment of the present formula is to bring an alternative in the control of this pathology as well as the other pathologies associated with metabolic syndrome.

Due to the exponential growth of alternative treatments with medicinal plants and nutraceuticals in inflammatory diseases, the examples and embodiments disclosed herein enable the method of treating with a composition including cannabinol (CBD), retinyl palmitate (RP), tributyrin (TB) and beta-caryophyllene (BC) which showed specific markers that control both the biochemical cascade of inflammation and the oxidative processes formed by radicals, among them PPAR-gamma, NF-kB, antioxidant activity, SOD2, pro-caspase-1, and glucose uptake. The therapeutic potential of these supplements has so far only been examined separately, a single antioxidant can hardly withstand oxidative damage in organs that have chronic inflammation.

The composition treats diseases related to metabolic syndrome with ingredients with low side effects and high pharmacological capacity in the control of inflammatory processes, high formation of free radicals and protection against apoptosis. In relation to previous applications, the ingredients were tested in a synergistic experiments, comparing with the ingredients in isolated form. As some ingredients have the ability to both interact with cell receptors as agonists and potentiate certain enzymes, the disclosed examples show that when these ingredients are administered concurrently, the results are superior when compared to isolated ingredients. As stated above, the therapeutic potential of these supplements has so far only been examined separately, with results that are quite satisfactory. However, our hypothesis is based on the fact that a single phytochemical ingredient can hardly be efficient enough to control oxidative stress and the inflammatory cascade formed in organs that present in pathologies such as metabolic syndrome. The disclosed composition and method comprises a synergistic approach as an alternative therapy showed support when the tests were investigated for the therapeutic potential in muscle and liver cells, especially when these cells were subjected to TNF-alpha treatment, which supports the hypothesis that a synergy of ingredients with concomitant metabolic activities can make a difference in certain pathologies when compared to the ingredients alone.

The composition has the best results when tested synergistically and the method of treatment administers the composition in a formula as an alternative therapy in treatments associated with metabolic syndrome. The ingredients can modulate the immunoinflammatory processes in various organs and serve as an alternative therapy in cases of drugs that trigger serious side effects. Patients with hepatic steatosis, fibromyalgia, type 2 diabetes can obtain promising results with the use of this conjugated formula. However, clinical studies in humans are needed to verify the significance of the results.

In developing nutraceuticals and/or phytochemicals, the possibilities are limitless. Thus, tests with conjugated formulas can be reinvented in terms of doses as well as possible other ingredients that can be added to the formula, but always within the concept of what was used, such as the combination of carotenoids, cannabinoids, terpenoids and butyrate. This would not limit the possibility of substituting any ingredients if necessary. However, first there is a need to know the pharmacodynamics and pharmacokinetics of the product in rats and then move to human studies. Studies in humans can be observed with specific markers, such as glycated hemoglobin (HbA1c), C-reactive protein (CRP), erythrocyte sedimentation value (ESR), Interleukin-6 (IL-6), tumor necrosis factor alpha (TNF-a) and others. In this embodiment, all the benefits and adverse effects of the proposed combinations would be understood.

Inflammatory processes are triggered by a nuclear protein called Nuclear Factor kappa Beta (NF-kB). When the expression of this protein is high, it activates other proteins that act as an inflammatory cascade, mainly interleukins, which can cause serious tissue damage. Normally these processes occur by metabolic changes in relation to oxidative stress. High ROS formations act as a trigger on NF-kB, causing, in many cases, the immune system to attack the tissue itself. The formula activates some nuclear receptors, mainly PPAR-alpha. This receptor, when activated, has the ability to signal and decrease NF-kB activity, thus turning off the inflammatory cascade. The formula and composition controls the levels of oxidative stress.

The formula and composition increases antioxidant processes, among them increasing the activity of the enzyme superoxide dismutase 2 (SOD2). Once oxidative stress is controlled, inflammation is controlled as a result, helping cells to re-enter homeostasis. Therefore, the formula and composition include ingredients that act synergistically in relation to both inflammation control and oxidative stress, especially on liver cells. The combinations of CBD, BC and TB, as well as RP and BC, showed remarkable results in increasing antioxidant activity, reducing inflammatory processes and controlling apoptosis. These results support and enable the composition in the treatment of liver-related diseases, such as hepatic steatosis, non-alcoholic liver cirrhosis and even in the control of tumors.

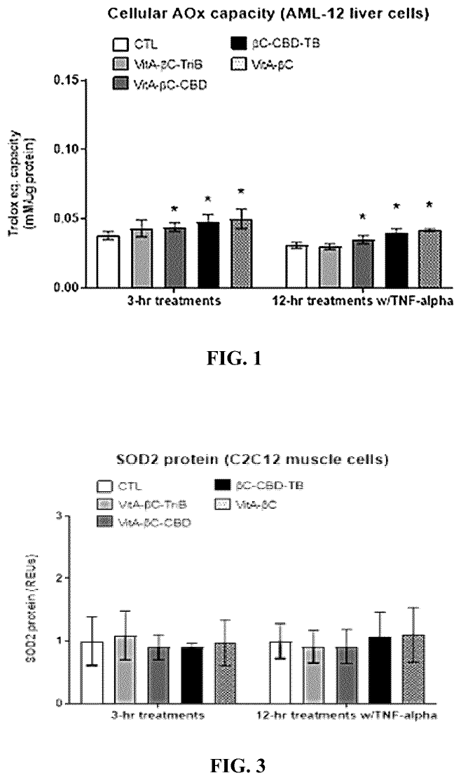

Considering that CBD mainly interacts not only with cannabinoid receptors but with a vast number of both nuclear and membrane receptors, the best synergistic compound partners in terms of biochemical activity to work with CBD. With this, some terpenes, as in the case of BC, a specific type of vitamin A RP as well as TB make perfect synergistic compound partners with CBD in the action of certain proteins/enzymes. This synergistic compound activity usually occurs because the receptors are either monomer (only one receptor), homodimers (two receptors of the same nature), or heterodimers (two receptors of different nature that act simultaneously). This means that at a given time, the ingredient can act as a single agonist of a particular receptor, as in the case of the monomer, or as a dual agonist, being a simultaneous agonist with two molecules in a homodimer. However, this does not happen in a heterodimer, which are two receptors of different nature that need agonists of different nature for an efficient action. These specific ingredients act simultaneously on different parts of the cells. Upon closer investigation, some combinations were more significant in inflammation control processes than others, as well as in antioxidant activity, as shown in FIG. 1.

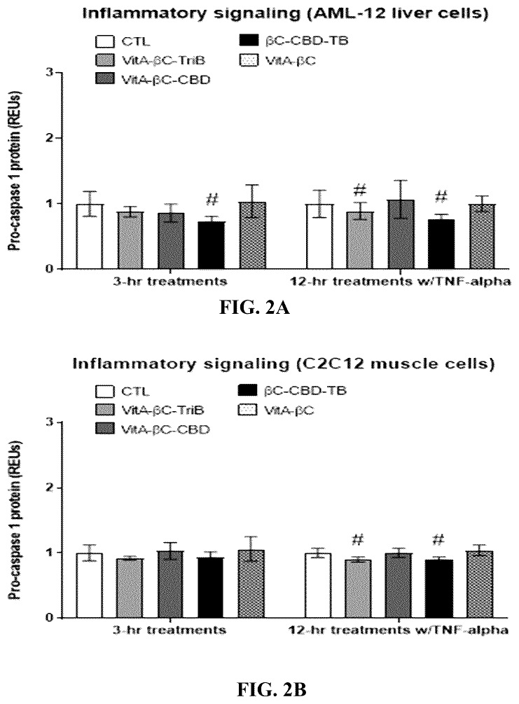

In some embodiments, the concomitant use of certain ingredients is effective when placed in a clinical trial. One of the most expressive results was the control of caspase-1 in liver cells evaluated in the study, mainly in cells that were exposed to TNF-alpha, as shown in FIGS. 2A-2B. Inhibiting cell apoptosis mechanisms is used to treat patients with liver disease who are on the verge of needing a transplant.

The composition controls inflammation and oxidative stress together with the control of apoptosis treats patients with metabolic syndrome with the combination of CBD, BC and TB. The RP with BC formula showed significant results in relation to increased antioxidant capacity and decreased inflammatory processes was. The RP with BC formula showed efficiency in controlling NF-kB, and thus reduced inflammatory processes in this type of tissue. Vitamin A with BC is synergistic with CBD, BC and TB in relation to activity of the PPAR-gamma DNA binding receptor. However, the composition of RP with BC significantly increased the activity of superoxide dismutase 2 (SOD2), as shown in FIG. 3. The efficiency of this enzyme in removing reactive oxygen is fundamental for the control of oxidative processes originating from ROS.

The formula and composition is a potential immunomodulator in inflammatory processes as well as in the control of ROS. The ingredients are presented as follows in two different formulas: Formula 1 comprising Butyric Acid 200 mg, Cannabidiol 30 mg, and Beta-Caryophyllene 10 mg. Formula 2 comprising Sodium Butyrate 175 mg, Beta-caryophyllene 15 mg, Vit A from retinyl palmitate 900 mcg, Beta-carotene 450 mcg, Lutein 2 mg, Zeaxanthin 400 mcg, Astaxanthin 2 mg, Lycopene 8 mg.

The ingredients of the formulas 1 and 2 can be changed both in terms of dosages and in the ingredients themselves, especially in terms of the action of carotenoids. Another important factor in relation to the formula is bioavailability. Using future techniques to improve bioavailability in relation to ingredients could be adopted. This is due to the adequacy of the effectiveness of the formula itself in future clinical trials to arrive at an ideal formula with regard to the treatment of pathologies related to the metabolic syndrome.

### Dose

In some embodiments, a pharmaceutical composition provided herein may include 0.1 mg/ml to 40 mg/ml of beta-caryophyllene. In some embodiments, the conjugated formulation may be admixtures of the bioactives. For example, a pharmaceutical composition provided herein may comprise 0.1 mg/ml to 30 mg/ml, 0.5 mg/ml to 30 mg/ml, 1 mg/ml to 30 mg/ml, 0.1 mg/ml to 25 mg/ml, 0.5 mg/ml to 25 mg/ml, 1 mg/ml to 25 mg/ml, 0.1 mg/ml to 20 mg/ml, 0.5 mg/ml to 20 mg/ml, 1 mg/ml to 20 mg/ml, 0.1 mg/ml to 15 mg/ml, 0.5 mg/ml to 15 mg/ml, 1 mg/ml to 15 mg/ml, 0.1 mg/ml to 10 mg/ml, 0.5 mg/ml to 10 mg/ml, 1 mg/ml to 10 mg/ml, 0.1 mg/ml to 5 mg/ml, 0.5 mg/ml to 5 mg/ml, 1 mg/ml to 5 mg/ml, 0.1 mg/ml to 2 mg/ml, 0.5 mg/ml to 2 mg/ml, 1 mg/ml to 2 mg/ml, 2 mg/ml to 40 mg/ml, 2 mg/ml to 30 mg/ml, 2 mg/ml to 25 mg/ml, 2 mg/ml to 20 mg/ml, 2 mg/ml to 15 mg/ml, 2 mg/ml to 10 mg/ml, 2 mg/ml to 5 mg/ml, 5 mg/ml to 40 mg/ml, 5 mg/ml to 30 mg/ml, 5 mg/ml to 25 mg/ml, 5 mg/ml to 20 mg/ml, 5 mg/ml to 15 mg/ml, 5 mg/ml to 10 mg/ml, 10 mg/ml to 40 mg/ml, 10 mg/ml to 30 mg/ml, 10 mg/ml to 25 mg/ml, 10 mg/ml to 20 mg/ml, 10 mg/ml to 15 mg/ml, 15 mg/ml to 40 mg/ml, 15 mg/ml to 30 mg/ml, 15 mg/ml to 25 mg/ml, 15 mg/ml to 20 mg/ml, 20 mg/ml to 40 mg/ml, 20 mg/ml to 30 mg/ml, 20 mg/ml to 25 mg/ml, 25 mg/ml to 40 mg/ml, 25 mg/ml to 30 mg/ml, 30 mg/ml to 40 mg/ml, or between 5 mg/ml and 125 mg/ml of beta-caryophyllene.

In some embodiments, a pharmaceutical composition provided herein may comprise at least 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 0.45 mg/ml to 4.5 mg/ml of Vitamin A.

In some embodiments, a pharmaceutical composition provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, or between 50 mg/ml and 5000 mg/ml of tributryin.

In some embodiments, a pharmaceutical composition provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 400 mg/ml and 4000 mg/ml of CBD.

In one embodiment, Vitamin A, beta-caryophyllene, and tributryin may be at a ratio of about 0.45 to about 4.5 mg of Vitamin A to: about 5 mg to about 125 mg of beta-caryophyllene to about 50 mg to about 5000 mg of tributryin.

In another aspect, the present invention encompasses a method for treating an inflammatory condition or disorder in a mammalian subject (preferably, but not exclusively, a mammalian subject) in need of such treatment, wherein said method comprises the administration (systemically, topically or by a combination of routes) of a composition of the present invention.

The present invention further provides a conjugated formulation for use as medicament or other therapeutic entity (such as ‘herbal remedy’, ‘food supplement’ and the like) in the treatment of an inflammatory condition. In one embodiment of this aspect, said composition is provided for use as a medicament or other therapeutic entity in the treatment of a systemic or topical or mucosal inflammatory disorder.

The present invention further provides the use of a pharmaceutical composition as disclosed herein for the preparation of a medicament. In some embodiments, this aspect of the invention relates to the use of a conjugated formulation as disclosed herein in the preparation of a medicament for use in the treatment of an inflammatory condition or disorder.

The present invention unexpectedly found that 1-beta-caryophyllene plus cannabidiol and tributyrin; 2-Retinyl palmitate (Vit A) plus beta-caryophyllene and cannabidiol; 3-Retinyl palmitate (Vit A) plus beta-caryophyllene controlled inflammation markers, apoptosis as well as enhance cellular antioxidant activity and a synergistic reduction of oxidative stress using a conjugated formulation comprising a combination of beta-caryophyllene, CBD, and tributryin as well as the conjugated formulation of Vitamin A, beta-caryophyllene, and CBD, as well as the conjugated formulation of Vitamin A and beta-caryophyllene. Surprisingly, the efficacy of this combination in inhibiting inflammatory mediators is comparable to the efficacy of DMSO, as will be shown in the experimental results presented hereinbelow.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, toffee, chocolate or cookie. In certain embodiments, the tablet or pellets are an immediate release or slow or controlled release dosage forms. In certain embodiment the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the cannabinoids at the distal intestine and may also release part of the cannabinoids in the intestines for systemic absorption and part of the cannabinoids at the colon for local colonic pharmacological effect.

In certain embodiments, the conjugated formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or otic or oral cavity or vaginal or rectal or uterine dosage form.

In certain embodiments, any one of the compositions described above, or any one of the dosage forms described above, is for use in a method of treating inflammation symptoms or disorders.

Preferred dosage forms include, but are not limited to, any liquid or semi solid or solid dosage form. The composition may be formulated in a medicament by preparing a topical or mucosal or oral delivery system. The topical delivery system may be in form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the composition further comprises one or more excipients selected from the group consisting of solvents, stabilizers, suspending agents, emulsifiers, release modifying, targeting and viscosity agents and combinations thereof.

In some embodiments, the composition of the present invention is formulated as a dosage form selected from the group consisting of a liquid, a suspension, an emulsion, a foam, a spray, a liposome, a semi-solid, a cream, an ointment, a patch, a particulate formulation, a granulate, a micro-particulate formulation, a nano-particulate formulation, a solid dosage form, a tablet, a capsule, an orally-disintegrable capsule, a mouth wash and an adhesive buccal tablet.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Design of the Formula with the Synergies in Combating Inflammatory Processes

The project was developed for clinical studies in muscle and liver cells. The study was carried out in muscle and liver cell culture as follows:

### Treatments and Cells

This report outlines treatments with one of five compounds: 1) Control or “vehicle” (DMSO only); 2) CBD (95 ng/mL); 3) Vitamin A, or “VitA” (1.2 μM); 4) Tributyrin (10 μM); and 5) Beta-caryophyllene (100 ng/mL).

There were two different treatment experiments including: Cells treated with compounds 1-5 above for 3 hours. Cells treated with compounds 1-5 above for 12 hours with a concomitant TNF-alpha treatment (15 ng/mL). Cell treatments were performed using C2C12 myotubes (muscle cells), and AML-12 cells (liver cells).

There are also combination treatments (phase II), where the following combinations were tested: 6) Control or “vehicle” (DMSO only); 7) Vitamin A, or “VitA” (1.2 μM)+Beta-caryophyllene (100 ng/mL)+Tributyrin (10 μM); 8) Vitamin A, or “VitA” (1.2 μM)+Beta-caryophyllene (100 ng/mL)+CBD (95 ng/mL); 9) Beta-caryophyllene (100 ng/mL)+CBD (95 ng/mL)+Tributyrin (10 μM); 10) Vitamin A, or “VitA” (1.2 μM)+Beta-caryophyllene (100 ng/mL).

As with single ingredients, two different treatment experiments occurred with these combos including: 1) Cells treated with compounds 1-5 above for 3 hours; and 2) Cells treated with compounds 1-5 above for 12 hours with a concomitant TNF-alpha treatment (15 ng/mL)

### General Culture Methods

All cells were grown to confluence using cell-specific media and additives.

The day of treatment, culture media was spiked with the appropriate ingredients (these are termed “treatment media”).

After either the 3-h or 12-h treatments described above, treatment media was removed, cells were rinsed with 1× phosphate-buffered saline, and general cell lysis buffer (Tris-based with detergent) was used to lyse cells.

Cells were scraped off the plate with a specific type of rubber in the lysis buffer, the slurry was placed in 1.7 mL tubes, and lysates were frozen at −80° C. until the respective assays discussed below.

There are n=5-6 replicates per treatment condition.

### Molecular Markers Assessed

Total antioxidant capacity was assessed using a Cu2+ ion conversion assay. Notably, all data was normalized to total protein loaded in the assay.

iNOS activity assays were performed using an enzymatic kit. Notably, all data was normalized to total protein loaded in the assay. This assay yielded low-to-no signal for most treatments; thus this was not included in the report.

PPARγ DNA binding activity was assessed using a plate that was pre-coated with a PPAR-gamma consensus binding sequence. Thereafter, antibody-based methods were used to assess the amount of bound transcription factor, and data were normalized to total protein loaded in the assay.

Cellular glucose was assayed using a colorimetric glucose assay kit that implemented the glucose oxidase enzyme reaction.

SOD2, SOD1, phosphorylated p65/NF-KB, pro-caspase 1, and NLRP3 were assayed using Western blotting methods. Notably, NLRP3 yielded low-to-no signal for most treatments; thus this was not included in the report.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method for immunomodulation in inflammatory processes and the control of reactive oxygen species, comprising:
a. providing a composition comprising Vitamin A, beta-caryophyllene (BCP), Cannabidiol, and butyrate or butyric acid in form of tributyrin; and
b. administering an effective amount of the composition to the subject to act on the subject's inflammatory condition.

2. A method for immunomodulation in inflammatory processes and the control of reactive oxygen species, comprising:
a. providing a composition comprising Vitamin A, beta-caryophyllene (BCP), Cannabidiol, butyrate or butyric acid in form of tributyrin, Beta-carotene, Lutein, Zeaxanthin, Astaxanthin, and Lycopene; and
b. administering an effective amount of the composition to the subject to act on the subject's inflammatory condition.
